(19) United States
(12) Patent Application Publication
(10) Pub. No.: US 2024/0090519 A1
(43) Pub. Date: Mar. 21, 2024

(54) NON-HYGROSCOPIC CURING AGENTS IN A GRANULAR FORM

(71) Applicant: INVENTIA HEALTHCARE LIMITED, Andheri (East) Mumbai (IN)

(72) Inventors: Vishal SHAH, Vile Parle (West), Mumbai (IN); Rajat SHAH, Vile Parle (West), Mumbai (IN); Sanjay SINGH, Kandivali East, Mumbai (IN)

(21) Appl. No.: 18/001,567

(22) PCT Filed: Jun. 8, 2021
(86) PCT No.: PCT/IB2021/055012

§ 371 Date: Dec. 12, 2022

(30) Foreign Application Priority Data
Jun. 10, 2020 (IN) 202021024271
Apr. 27, 2021 (IN) 202121019311

(51) Int. Cl. A23B 4/027; A23B 4/20; A23B 4/24

(52) U.S. Cl. CPC A23B 4/027; A23B 4/20; A23B 4/24

(57) ABSTRACT

The present invention provides a solid, non-hygroscopic curing agent in a powdered/granular form easy to use, handle and optionally reconstitute, comprising a plant-based nitrite derived from a concentrated liquid extract obtained from plant material selected from Beet, radish, onion, celery and chicory leaf. In particular, it relates to a plant-based nitrite derived from a concentrated liquid extract obtained from plant material celery. The non-hygroscopic powder/granular agent has an excellent solid-state stability, longer shelf-life, easy to package and store. It can be used to preserve or cure meat or meat products and also has other applications in the food industry.

### BACKGROUND OF THE INVENTION

The preservation of meat and meat products has been practiced for many years in a variety of ways, such as by smoking, treatment with or nitrite, or combinations thereof. In one method for preparing cured meat and meat products, the meat or meat product desired to be cured is exposed to a nitrate-containing substance. It is prepared by addition of bacteria or other organisms capable of converting the nitrate to nitrite to ferment the nitrate to nitrite as in the case of vegetable juice concentrates like those of cabbage, beet, celery, radish, onion and chicory leaf. The presence of nitrite helps in curing of meat to give the meat a distinct colour, retention of freshness and flavour, in addition to preventing the growth of harmful microorganisms. Advancements in this field have resulted in readily available nitrite containing liquid concentrates from various plant materials such as celery, beet, cabbage, radish, onion and chicory leaf. However, a disadvantage is that liquid agents are difficult to use, transport and when concentrated extracts are converted to a powdered form, they are unstable. The major problem with such solid forms of concentrated liquid extracts obtained by processes such as spray drying, or drum drying is the unstable hygroscopic form that is obtained which changes its characteristics when stored or exposed to open atmosphere.

FIG. 1 demonstrates this instability wherein a commercially available celery extract in powdered form turns into an unstable black mass within 30 minutes when exposed to room atmosphere.

There is need in the art for a solid, non-hygroscopic curing agent in a powdered form easy to use, handle and optionally reconstitute, comprising a plant-based nitrite derived from a concentrated liquid extract obtained from plant material selected from beet, radish, onion, celery and chicory leaf or combinations thereof.

The present invention is directed towards a non-hygroscopic powder/granular agent having an excellent solid-state stability, longer shelf-life, easy to package and store. It can be used to preserve or cure meat or meat products and other applications in the food industry.

US 20150225683 relates to a powdered vinegar comprising partly neutralized vinegar, e.g. neutralized with a sodium and/or potassium hydroxide, and free acid. The invention also provides a process for producing such a powdered vinegar and the use of this powdered vinegar in a foodstuff or a beverage, for example as a preservative.

US 20170107468 relates to an invention in the field of liquid, concentrated food preservation products, in particular concentrated vinegar products. The present invention provides a method of producing highly concentrated vinegar, enabling the production of food-grade liquid preservation products comprising high amounts of vinegar. Such liquid preservation products and their uses are also encompassed by the invention.

WO 2019075016 is an invention that discloses an antimicrobial powder, which includes a low molecular weight antimicrobial organic acid and a hygroscopic ingredient, prepared according to a process wherein the components are combined in a solution to form a liquid slurry composition prior to drying to form a powder, such that drying the liquid slurry composition involves co-drying the components. The low molecular weight antimicrobial organic acid in the antimicrobial powder can be present in a crystal phase of both anhydrous and hydrate forms. The antimicrobial powder exhibits excellent properties, such as shelf stability, without the requirement of encapsulating agents. Methods for preparing the antimicrobial powders as well as applications of the antimicrobial powders in food and beverage products are also disclosed.

### SUMMARY OF THE INVENTION

The present inventors have developed a solid, non-hygroscopic curing agent in a powdered/granular form easy to use, handle and optionally reconstitute, comprising a plant-based nitrite derived from a concentrated liquid juice concentrate obtained from plant material selected from celery. The non-hygroscopic powder/granular product has an excellent solid-state stability, longer shelf-life, easy to package and store. It can be used to preserve or cure meat or meat products and other applications in the food industry.

The method according to the present invention comprises the steps of:
a. Providing a concentrated liquid juice concentrate/extract having around 40% w/w of solids, obtained from plant material selected from celery, a water insoluble inorganic carrier preferably in the form of precipitated silica and or a salt to carry the liquid juice concentrate/extract, starch/modified starch or starch-based derivatives; cellulose derivatives and optionally an additive or carrier which may also function as a stabiliser to facilitate the formation of a free-flowing non-hygroscopic final product
b. Loading of the concentrated liquid extract obtained from the plant material celery onto the water insoluble inorganic carrier preferably precipitated food grade silica/silica or salts of silica or other inorganic moiety using a suitable process like blending, spray granulation, mixing, fluid bed processing/granulation & spray drying or a combination of these processes.
c. Drying the final product to a desired moisture content using a suitable process
d. Cooling the product to room temperature before packing

The present process offers the advantage that it yields a stable, non-hygroscopic, free flowing powdered/granular curing agent that can suitably be used as a preservative in e.g., processing and curing of meat products.

Another aspect of the invention relates to powdered/granular curing agent that is obtained by the aforementioned process.

Yet another aspect of the invention relates to the use of the powdered/granular curing agent in the food industry.

### DESCRIPTION OF FIGURE

FIG. 1 demonstrates instability wherein a commercially available celery extract in powdered form turns into an unstable black mass within 30 minutes when exposed to room atmosphere.

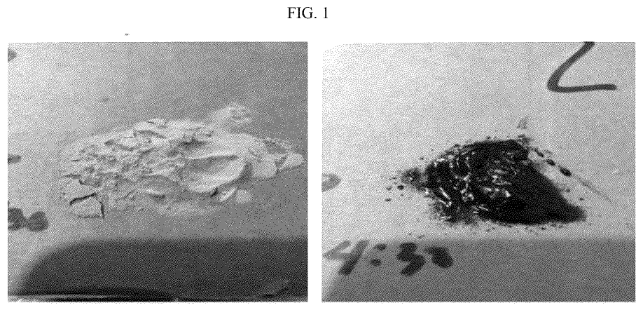

FIG. 2 is a flow chart of a process for manufacturing the curing agent described herein.

FIG. 3 is a table with observations of a reference product and the exemplified non-hygroscopic celery juice concentrate powder in an open pack at 40° C. and 75% RH initially and after 7 and 14 days.

### DETAILED DESCRIPTION OF THE INVENTION

Accordingly, a first aspect of the invention concerns a process of producing a non-hygroscopic powdered curing agent comprising the steps of:
a) providing a concentrated liquid juice concentrate/extract obtained from celery plant material which comprises the curing agent, preferably nitrite
b) optionally dissolving/dispersing a starch/modified starch or starch-based derivatives or derivatives of cellulose in the concentrated liquid juice
c) loading of this liquid juice concentrate/extract onto a water insoluble inorganic carrier preferably precipitated food grade silica/silica, or its salts or salts of inorganic metals, by using a suitable process to get a uniform distribution of the liquid extract on the carrier.
d) processing the mixture obtained in the previous steps using various processes like drying and optionally blending, mixing to obtain a suitable or desired water content of the final product
f) Cooling of the final product to room temperature
g) Sifting and sizing of the final non-hygroscopic powdered/granular curing agent followed by packing

The term “non-hygroscopic granular/powdered curing agent” as used herein refers to a solid curing agent preferably in the form of granules or powder or a mixture thereof that does not take up significant amounts of moisture from the atmosphere when exposed, such that there is no liquefaction or change in the solid form of the curing agent to semi solid wet mass.

The term “liquid juice concentrate/extract obtained from a plant material” as used herein refers to an aqueous liquid juice concentrate/extract that may be in a concentrated or dilute form comprising the curing agent preferably nitrite and may have a solid content varying from about 20 to 60% w/w, preferably 30 to 45% w/w, most preferably from 35 to 40% w/w, obtained from the plant material of celery. The nitrite content in the liquid concentrate may vary from 9 to 30 mg/ml of the liquid concentrate and can be adjusted during the formulation based on the desired nitrite content in the final product.

The term “concentrated liquid juice/extract-obtained from a plant material” as used herein refers to a concentrated aqueous liquid extract that contains the curing agent preferably nitrite at a solid content of about 40% w/w preferably corresponding to a nitrite concentration of approximately 10,000 to 60,000 ppm and is obtained from plant material selected from celery.

The term “water insoluble inorganic carrier” as used herein refers to an inorganic material like precipitated food grade silica/silica or silica present in any insoluble form or water insoluble salts of an inorganic metal.

The term “uniform distribution of liquid”, refers to a process used for loading the liquid onto the water insoluble silica and covers the processes known in the art including blending, pouring spraying, atomized delivery etc.

The term “granulation” refers to a process of converting the powder into granules employing a suitable process with the aid of equipment's like a rotary mixer granulator, blender, coater, spray granulation, spray drying or a combination of processes mentioned.

The term “co-processing” refers to multiple operations of unit processes performed to achieve the desired result and may include processes like blending, mixing and drying in series or in parallel.

According to a preferred embodiment of the invention, the water insoluble inorganic silica is preferably sifted through a 20 to 100 mesh sieve before processing. The concentrated liquid juice containing nitrite as the curing agent, obtained from plant material may be in the form of a suspension. The ratio of the water insoluble precipitated silica to the concentrated liquid juice/extract may vary from 1:1 to 1:20, more preferably the ratio of the water insoluble precipitated silica to the solid content of concentrated liquid juice/extract may vary from 1:0.5 to 1:10. These ratios are subjected to inherent concentration of Sodium Nitrite in Celery Juice concentrate/extract.

The starch/modified starch, starch derivatives or cellulose derivatives which belong to the group of polymeric carbohydrates or polysaccharides are used as filler, binder, diluent and as an excipient in the composition. The concentration of starch/modified starch, starch derivatives, cellulose derivatives may vary from 2 to 15% w/w of the final product on a w/w basis, more preferably from 4 to 12% w/w of the final product.

The process of manufacture involves the following steps:
Weighing: All raw materials are weighed separately
Solution Preparation: The starch/modified starch/starch derivative is dissolved in the Celery juice concentrate (containing 40% w/w on solid basis) under stirring for a period of 12 to 30 minutes and the solution is passed through a mesh of 20-80 preferably 40 mesh, which also acts a s a filtration and homogenisation step.
Granulation in Fluid Bed Processor: Precipitated silica is loaded onto a fluid bed processor and the solution obtained in the previous step is sprayed onto the precipitated silica under fluidisation. The inlet air temperature may be maintained at 45° C. to 90° C. and process continued till all solution is sprayed taking care that the product temperature is maintained between 25° C. to 38° C. On completion of the process the granules are dried in the fluid bed processor at an inlet temperature from 45° C. to 60° C., till a loss on drying (LOD) value of less than 15% w/w is achieved. The finished granules may then be unloaded and packed in suitable polythene bags.

The process steps can be depicted as below:

Process Steps

| | |
|---|---|
| Weighing | Weighing of All Raw Materials Separately |
| Solution | Dissolve Modified starch and/or suitable |
| Preparation | binder (cellulose derivatives) in Celery |
| | Juice concentrate/extract, under stirring |
| | for 12 to 30 minutes |
| Filtration | Pass the solution through sieve 40 mesh |
| Granulation | Load the Precipitated Silica in a Fluid |
| in FBP | Bed processor |
| | Top Spray the above filtered celery |
| | juice concentrate on silica |
| | Inlet temp: 45° C. to 75° C. |
| | Product temp: NMT 38° C. |
| | (25° C. to 38° C.) |
| Drying | Dry the material till LOD < 15% w/w |
| | With Inlet temp: 45° C. to 60° C. |
| Packaging & | Unload the Finished granules and Pack |
| Labelling | in Polybag and Perform the labelling |

According to another preferred embodiment of the invention, the water insoluble inorganic salt is preferably sifted through a 20 to 100 mesh sieve before processing. The concentrated liquid extract containing nitrite as the curing agent, obtained from plant material may be in the form of a suspension. The ratio of the water insoluble inorganic salt to the concentrated liquid extract may vary from 1:3 to 1:5. The water insoluble inorganic salt is loaded onto a suitable jacketed mixer/blender which is pre-heated to a temperature varying from 10° C. to 100° C. The concentrated liquid extract which is in the form of a solution or suspension is added to the water insoluble inorganic salt in the form of a fine or coarse liquid spray on the agitated mass in the blender/mixer containing the inorganic salt at a speed which may vary from 5 rpm to the maximum speed based on the process and final granules desired. The spray may be generated using a suitable spraying system such that the top surface of the salt is wetted continuously. On complete addition of the liquid extract the granulation equipment is run at suitable speeds and parametric conditions and granulate characteristics are ascertained. In case of irregularity and non-consistency a binder such as HPMC or starch may be further used, and the mass granulated to obtain consistency and uniformity of granules. The granulator is stopped, and material is raked to ensure uniformity of sample including a top bottom raking. In case post raking granule uniformity is not observed a stabiliser such as edible oil and lecithin may be added, and the granules further processed with a granulation cycle of forward and reverse blades at a suitable rpm. Once granulation is completed, silicon dioxide, talc, calcium salts previously sifted through 30 to 100 mesh sieves is weighed and added to this granulated mass as a glidant in concentrations ranging from 0.001 to 0.05% w/w. Then the entire mass is sifted through a sifter from 20 to 100 mesh. After this the blending cycle of forward and reverse blades at a similar rpm and speed is repeated. This completes the granulation process. The granulated mass is dried at 15° C. to 150° C. in Fluidized Bed Drier and drying continued till a desired moisture level is achieved. Once moisture level is achieved the powdered mixture is allowed to cool to room temperature.

The process of manufacture involves the following steps:
a) providing a concentrated liquid extract obtained from celery plant material which comprises the curing agent, preferably nitrite
b) loading of this liquid extract onto a water insoluble inorganic carrier preferably in the form of a salt, by using a suitable process to get a uniform distribution of the liquid extract on the water insoluble inorganic carrier.
c) granulation of this mass after loading of the liquid extract using a suitable machine like a granulator, blender,
d) addition of a silica-based additive/carrier along with binders and stabilizers post-granulation to facilitate the flow of the granules for subsequent processing.
e) co-processing the mixture obtained in the previous steps using various processes like blending, drying, mixing to obtain a suitable or desired water content of the final product
f) Cooling of the final product to room temperature
g) Sifting and sizing of the final non-hygroscopic powdered/granular curing agent followed by packing.

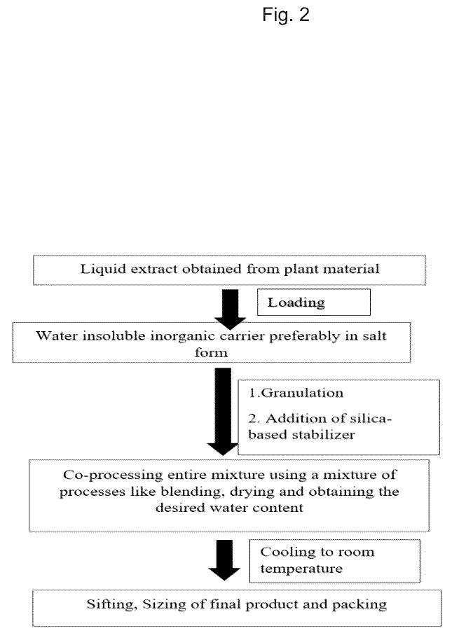

The process steps can be depicted as shown in FIG. 2.

### EXAMPLES

The invention using a process as depicted in FIG. 2A using food grade silica/silica is further illustrated by the following non-limiting examples.

### Example 1: Preparation of Non-Hygroscopic Free Flowing Celery Powder with Around 22,500 ppm of Nitrite Content

A batch size of around 614 gm was manufactured based on the composition details as tabulated below:

| Ingredient | Qty/Batch (Gm) |
|---|---|
| Celery Juice Concentrate* | 800 (320^{$}) |
| Precipitated Silica | 248 |
| Modified starch | 46 |
| (Sodium starch | |
| octenyl succinate | |

Note:
*celery juice concentrate, contains 40% solid and Active Sodium nitrite is around 18000 ppm
^{$}On solid basis at 40% of 800 gm of juice/liquid concentrate works out to 320 gm.
Total weight - 614.00 gm (As on solid basis)

The manufacturing process described in general in FIG. 2A was broadly followed with very minor modifications if needed to arrive at a product which was non-hygroscopic, granular, and free flowing.

To evaluate the non-hygroscopicity the product prepared as described above was subjected to exposure to the following conditions in open petri-dishes:
a. Exposure in open petri dish in Accelerated chamber 40° C./75% RH: LOD was monitored, and it was observed that product was free flowing. Exposure lasted to about 5 hours. Though the product picked up moisture it was still free flowing.
b. Exposure in open petri dish exposed to open ambient atmosphere: LOD was monitored, and it was observed that product was free flowing for about 7 days in unpacked condition. Though the product picked up moisture it was still free flowing.

### Example 2: Preparation of Non-Hygroscopic Free Flowing Celery Powder with Around 27000 ppm of Nitrite Content

A batch size of around 950 gm was manufactured based on the composition details as tabulated below:

| Ingredient | Qty/Batch (Gm) |
|---|---|
| Celery Juice Concentrate* | 1600(640^{$}) |
| Precipitated Silica | 250 |
| Modified starch | 60 |
| (Starch Sodium | |
| Octenyl Succinate) | |

Note:
*celery juice concentrate, contains 40% solid and Active nitrite is around 16500 ppm
^{$}On solid basis at 40% 800 gm of juice/liquid concentrate works out to 640 gm.
Total weight - 950.00 gm (As on solid basis)

The manufacturing process described in general in FIG. 2A was broadly followed with very minor modifications if needed to arrive at a product which was non-hygroscopic, granular, and free flowing.

To evaluate the non-hygroscopicity the product prepared as described above was subjected to exposure to the following conditions in open petri-dishes:
a. Exposure in open petri dish in Accelerated chamber 40° C./75% RH: LOD was monitored, and it was observed that product was free flowing. Exposure lasted to about hours. Though the product picked up moisture it was still free flowing.
b. Exposure in open petri dish exposed to open atmosphere: LOD was monitored, and it was observed that product was free flowing for about 7 days in unpacked condition. Though the product picked up moisture it was still free flowing.

### Example 3: Preparation of Non-Hygroscopic Free Flowing Celery Powder with not Less than 15000 ppm of Nitrite Content

A batch size of around 130 gm was manufactured based on the composition details as tabulated below:

| Ingredient | Qty/Batch (Gm) |
|---|---|
| Celery Juice Concentrate* | 111(44.4^{$}) |
| Precipitated Silica | 60 |
| HPMC | 5 |
| Refined Sunflower | 19.7 |
| Oil with TBHQ | |
| Sunflower Lecithin Liquid | 0.98 |

Note:
*celery juice concentrate, contains 40% solid and Active nitrite is around 18000 ppm
^{$}On solid basis at 40% 800 gm of juice/liquid concentrate works out to 44.4 gm.
Total weight - 130.00 gm (As on solid basis)

The manufacturing process described in general in FIG. 2A was broadly followed with very minor modifications if needed to arrive at a product which was non-hygroscopic, granular, and free flowing.

The difference in manufacturing process was slightly different in the sense that a planetary mixer was loaded with precipitated silica and concentrated celery juice was added to this precipitated silica, followed by drying to achieve a desired LOD. HPMC was dissolved in water and added to above silica loaded celery to obtain granules followed by drying to get desired LOD. The granules obtained were sized if needed and loaded onto a planetary mixer. Sunflower lecithin was dissolved in sunflower oil to achieve a uniform oil phase with slight heat if needed and this oily mass was added to the HPMC-silica-celery granules to obtain a non-hygroscopic, non-dusty free flowing powder.

To evaluate the non-hygroscopicity the product prepared as described above was subjected to exposure to atmosphere in open petri-dishes and it was observed that up to 8 hours product was non-hygroscopic and free flowing.

The invention using a process as depicted in FIG. 2B using a water insoluble salt of silica or any other metal ion is further illustrated by the following non-limiting examples.

### Example 4: Preparation of Non-Hygroscopic Free Flowing Celery Powder/Granules with Around 70% w/w Celery (with not Less than 22,500 ppm of Nitrite Content)

A) Weighing of Celery Extract Liquid, calcium silicate and Silicon dioxide: Sift 900 to 1000 g of Ca silicate using sieve 60 to 100 mesh size. Weigh approximately 990 g sifted powder of Calcium silicate in a LDPE bag & close it securely. Shake vigorously the liquid celery extract container to ensure uniform dispersion of solid in liquid matrix. Measure a quantity of about 2500 ml of liquid celery extract from it in a clean container. Thus, the percentage of liquid celery extract to total batch size is around 70% w/v.
B) Loading of calcium silicate with mixing

Add sifted Calcium silicate into the blending machine which is previously heated from room temperature to a temperature varying from room 10° C. to 95° C.
C) Addition of Celery Extract Liquid

Add liquid celery extract by forming a fine to coarser size liquid spray on agitated mass of Calcium silicate at a speed varying from 5 rpm to a desired speed capable of forming good granules. Ensure that spray is added from all possible side to wet the top surface continuously.

D) Granulation
1. Run the machine at varying rpm of 5 to 90 for a time varying from 2 to 30 minutes in forward direction and reverse direction as per the process.
2. A binder such as HPMC or starch may be further used for granulation optionally to achieve better granule characteristics and properties, by further running the granulator at a suitable speed and time with binder solution being introduced into the granulator.
3. Post optional binder usage, check for granule characteristics and if need be, use a stabiliser such as edible oil and lecithin on the granules post binder granulation for stabilization of the granule surface.
4. Stop the machine to do raking of all parts of blender including the blades & dead ends.
5. Remove 300 g to 500 g of sample from the bottom & add it to the top mass surface & blend it for 5 minutes.
E) Addition of Silicon dioxide & co-sifting
1. Add 0.001 to 0.05% w/w of Silicon dioxide powder or talc previously sifted from 30 to 100 mesh sieve.
2. Mix using a suitable blender at an optimised rpm and time to achieve a uniform blend of granules with good flow properties.
1. Drying: The granulated mass is dried at v 15° C. to 150° C. in a fluidized bed drier till desired moisture level is attained.

F) Celery Extract Powder
1. Cool the powder to room temperature and send the sample for analysis of Quality parameters.

Further examples depicted below follow a substantially similar process flow as mentioned in FIGS. 2A/2B

### Example 5: Preparation of Non-Hygroscopic Free Flowing Celery Powder with Around 18,500 ppm of Nitrite Content

Composition:

| | Process | Raw | Content % | Quantity |
|---|---|---|---|---|
| Sr. No. | Step | Material | w/w | (kg) |
| 1 | Dry Mix | Calcium Silicate | 48.30 | 0.966 |
| 2 | Addition of | Celery Juice | 49.28 | 2.464 |
| | Celery | Concentrate* | | |
| | Concentrate | (Solid Content | | |
| | Juice | 40% w/w) | | |
| 3 | Silicon dioxide | Lubricant | 2.41 | 0.0482 |

| | | |
|---|---|---|
| Total | 100.00 | 2.000 |

Note:
*celery juice concentrate, contains 40% solid and Active nitrite is around 15000 ppm
^{#} May or may not be part of final product

Manufacturing Process: The Process is Described Briefly Here Under:

Weighing of Celery Juice Concentrate and Calcium silicate was done individually. The addition of the celery juice concentrate to calcium silicate was done by hand granulation. The ratio of water insoluble silicate to celery liquid extract is around 1:2.62. Powder was dried to moisture level NMT 5%. using hand granulation (as batch size was small) and the resulting granules were dried at 85° C. till LOD of less than 5% was achieved. Powder was sifted using 30 #. LOD and nitrite content were determined.

Conclusion: Product is found non hygroscopic till 1 week in unpacked condition.

Nitrite content: 18.480 ppm

Observations: White to off-white free flowing dusty powder having a bulk density of 0.26 g/cc

### Example 6: Preparation of Non-Hygroscopic Free Flowing Celery Powder with Around 18,000 ppm of Nitrite Content

Composition:

| | Process | Raw | Content % | Quantity |
|---|---|---|---|---|
| Sr. No. | Step | Material | w/w | (kg) |
| 1 | Dry Mix | Calcium Silicate | 43.50 | 0.705 |
| 2 | Addition of | Celery Juice | 49.40 | 2.000 |
| | Celery | Concentrate* | | |
| | Concentrate | (Solid Content | | |
| | Juice | 40% w/w) | | |
| 3 | Binder | HPMC (Binder) | 3.55 | 0.0575 |
| 4 | | Purified water | — | QS |
| | | for Binder | | |
| 5 | Stabiliser | Soya Lecithin | 3.55 | 0.0575 |
| | | Wax (Stabiliser) | | |
| 6 | | Edible vegetable | — | QS |
| | | oil for stabiliser | | |

| | | |
|---|---|---|
| Total | 100.00 | 1.620 |

Note:
*celery juice concentrate, contains 40% solid and Active nitrite is around 15000 ppm
^{#} May or may not be part of final product

Manufacturing process: The process is described briefly here under:

Binder Preparation: Add HMPC into purified water under continuous stirring, till HMPC gets completely dissolved.

Stabiliser Preparation: Add Soya Lecithin Wax into edible vegetable oil with continuous stirring till wax gets completely dissolved.

Dry mixing and Granulation: The ratio of water insoluble silicate to celery liquid extract is around 1:2.8. Load Calcium Silicate in Ribbon Mixer and dry mix at 20 to 50 RPM for 0.5 to 2.0 minutes. Slowly pour Celery Juice Concentrate manually at a suitable speed for around 2 to 10 minutes and continue mixing in both forward and reverse directions for a suitable time, each twice, with scrapping and raking of granulated mass intermittently. Stop ribbon mixer, scrape the material stuck on ribbon mixer strands, corners, and rake

Repeat granulation at higher speed of 70-120 RPM in forward and reverse direction for a suitable time and repeatedly each for 2 to 5 times to have uniformly mixed mass. Start mixer at a low speed of around 20 RPM and slowly add Soya Lecithin Wax solution (Stabiliser) over a time interval of around 2-5 mins. Perform additional mixing if needed to get uniformly mixed wet mass.

Dry the material at 100° C. jacket temperature for around 2 to 3 hours with a suitable ribbon rotation speed. Record L.O.D.

Sizing and drying: Unload material from ribbon mixer and pass this dried mass through 20#sieve manually. Load sifted material into Fluid Bed Equipment and carry out drying at inlet temperature of 100° C., till desired LOD is achieved (around 10% w/w).

Unload the material into suitable size container and sift. the material through 20 #sieve and record weight (approx. 1.6 kg)

Observation of unpacked granules to atmospheric condition for its non-hygroscopic behaviour with time

| | CELP-2005015 | Physical |
|---|---|---|
| Time in Hrs | % Moisture | observations |
| 0 | 4.65 | Non hygroscopic free flowing |
| 4 | 7.5 | Non hygroscopic free flowing |
| 24 (1 day) | 10.5 | Non hygroscopic free flowing |
| 96 (4 days) | 12 | Non hygroscopic free flowing |
| 168 (7 days) | 14.5 | Non hygroscopic free flowing |

Conclusion: Product is found non hygroscopic till 1 week in unpacked condition.
Nitrite content: 18750 ppm

During analysis it was observed that the powder was reported to be dusty during reconstitution in large quantities and hence it was decided to make the product granular and non-dusty.

### Example 7: Preparation of Non-Hygroscopic Free Flowing Celery Powder with Around 15,000 ppm of Nitrite Content

Composition:

| | Process | Raw | Content % | Quantity |
|---|---|---|---|---|
| Sr. No. | Step | Material | w/w | (gm) |
| 1 | Dry Mix | Calcium Silicate | 23.20 | 46.34 |
| 2 | Addition of | Celery Juice | 44.20 | 220.15 |
| | Celery | Concentrate* | | |
| | Concentrate | (Solid Content | | |
| | Juice | 40% w/w) | | |
| 3 | Dry Mix | Sunflower | 0.02 | 3.0 |
| | | lecithin | | |
| 4 | Dry mix | Partially | 31.25 | 62.50 |
| | | hydrogenated | | |
| | | vegetable fat | | |

| | | |
|---|---|---|
| Total | 100.00 | 200 (on |

solid basis)
Note:
*celery juice concentrate, contains 40% solid and Active nitrite is around 15000 ppm

Manufacturing process: The process was essentially similar to the process described in Example 2B but is given briefly here and the ratio of water insoluble silicate to celery liquid extract is more than 1:4.7

Weighing of Celery Juice Concentrate and Calcium silicate was done individually. The addition of the celery juice concentrate to calcium silicate was done by hand granulation.

Powder was dried to moisture level NMT 5%. Liquid Celery Juice concentrate was added again on dried powder mix regenerated earlier using hand granulation (as batch size was small) and the resulting granules were dried at 85° C. till LOD of less than 5% was achieved. Granules were sifted through 16 #. Granules were dried at 85° C. till moisture level achieves not more than 5%. Sunflower lecithin was added to hydrogenated vegetable fat and the granules were granulated using this hydrophobic mixture in a planetary mixer over a period of 5 to 10 minutes at a suitable speed. The final product after completion of the mixing in planetary mixer was sifted using 30 #. LOD and nitrite content were determined

Conclusion: Product is found non hygroscopic till 1 week in unpacked condition. Nitrite content: 16575 ppm

It was observed that addition of hydrophobic ingredient significantly reduced the dusting characteristic of the powder.

### Example 8: Preparation of Non-Hygroscopic Free Flowing Celery Powder with Around 24,800 ppm of Nitrite Content

Composition

| | Process | Raw | Content % | Quantity |
|---|---|---|---|---|
| Sr. No. | Step | Material | w/w | (gm) |
| 1 | Dry Mix | Calcium Silicate | 23.10 | 46.20 |
| 2 | Addition of | Celery Juice | 66.15 | 330.77 |
| | Celery | Concentrate* | | |
| | Concentrate | (Solid Content | | |
| | Juice | 40% w/w) | | |
| 3 | Dry Mix | Sunflower | 0.49 | 0.98 |
| | | lecithin | | |
| 4 | Dry mix | Partially | 10.20 | 20.40 |
| | | hydrogenated | | |
| | | vegetable fat | | |

| | | |
|---|---|---|
| Total | 100.00 | 200 (on |

solid basis)
Note:
*celery juice concentrate, contains 40% solid and Active nitrite is around 15000 ppm

Manufacturing process: The process was essentially similar to the process described in Example 2B, the ratio of water insoluble silicate to celery liquid extract is around 1:7, but is given briefly here:

Weighing of Celery Juice Concentrate and Calcium silicate was done individually. The addition of the celery juice concentrate to calcium silicate was done by hand granulation. Powder was dried to moisture level NMT 5%. Liquid Celery Juice concentrate was added again on dried powder mix regenerated earlier using hand granulation (as batch size was small) and the resulting granules were dried at 85° C. till LOD of less than 5% was achieved. Granules were sifted through 16 #. Granules were dried at 85° C. till moisture level achieves not more than 5%. Sunflower lecithin was added to hydrogenated vegetable fat and the granules were granulated using this hydrophobic mixture in a planetary mixer over a period of 5 to 10 minutes at a suitable speed. The final product after completion of the mixing in planetary mixer was sifted using 30 #. LOD and nitrite content were determined Conclusion: Product is found non hygroscopic till 1 week in unpacked condition. Nitrite content: 24825 ppm

### Example 9: Preparation of Non-Hygroscopic Free Flowing Celery Powder with Around 24,500 ppm of Nitrite Content

Composition:

| | | | | Powder |
|---|---|---|---|---|
| | Process | Raw | Content % | Quantity |
| Sr. No. | Step | Material | w/w | (gm) |
| 1 | Dry Mix | Calcium Silicate | 25 | 50.00 |
| 2 | Addition of | Celery Juice | 66 | 330.0 |
| | Celery | Concentrate* | | |
| | Concentrate | (Solid Content | | |
| | Juice | [symbol] % w/w) | | |
| 3 | Granulation | HPMC K100 LV | 2.5 | 5.0 |
| 4 | Dry Mix | Sunflower | 0.56 | 1.12 |
| | | lecithin | | |
| 5 | Dry mix | Normal sunflower oil | 11.22 | 22.44 |

| | | |
|---|---|---|
| Total | 100.00 | 200 (on |

solid basis)
Note:
*celery juice concentrate, contains [symbol] % solid and Active nitrite is around 15000 ppm

Manufacturing process: The process was essentially similar to the process described earlier and the ratio of water insoluble silicate to celery liquid extract is around 1:6.5, but is given briefly here:

Weighing of Celery Juice Concentrate and Calcium silicate was done individually. The addition of the celery juice concentrate to calcium silicate was done by hand granulation. Powder was dried to moisture level NMT 5%. Liquid Celery Juice concentrate was added again on dried powder mix regenerated earlier using hand granulation (as batch size was small) and the resulting granules were dried at 85° C. till LOD of less than 5% was achieved. Granules were created using binder preparation from HPMC & purified water. Wet mass sifted through 16 #. Granules were dried at 85° C. till moisture level achieves not more than 5%. Sunflower lecithin was added to hydrogenated vegetable fat and the granules were granulated using this hydrophobic mixture in a planetary mixer over a period of 5 to 10 minutes at a suitable speed. The final product after completion of the mixing in planetary mixer was sifted using 30 #. LOD and nitrite content were determined.

Conclusion: Product is free flowing for 3 days in an un packed condition & exposed to open atmosphere.

Nitrite content: 24750 ppm

### Example 10: Preparation of Non-Hygroscopic Free Flowing Celery Powder with Around 15,600 ppm of Nitrite Content

Composition:

| | | | | Powder |
|---|---|---|---|---|
| | Process | Raw | Content % | Quantity |
| Sr. No. | Step | Material | w/w | (gm) |
| 1 | Dry Mix | Calcium Silicate | 16.66% | 33.35 |
| 2 | Addition of | Celery Juice | 44.0% | 234.11 |
| | Celery | Concentrate* | | |
| | Concentrate | (Solid Content | | |
| | Juice | 36% w/w) | | |
| 3 | Granulation | HPMC K100 LV | 1.66% | 3.335 |
| 4 | Dry Mix | Sunflower | 0.373% | 1.12 |
| | | lecithin | | |
| 5 | Dry mix | Refined | 7.40% | 0.75 |
| | | sunflower oil | | |
| 2 | Dry Mixing | Magnesium citrate | 33.33% | 77.84 |

| | | |
|---|---|---|
| Total weight | 100% | 200.00 g |

Note:
*celery juice concentrate, contains 36% solid and Active nitrite is around 13000 ppm

Manufacturing process: The process was essentially similar to the process described in Example 4 and the ratio of water insoluble silicate to celery liquid extract is around 1:6.5, with the following additional step:

The celery extract powder blend and magnesium citrate were weighed individually. The Magnesium citrate was added to the celery extract powder blend while mixing in geometric proportions and the final mixture was sifted using 18 #sieve.

Nitrite content:15210 ppm

Moisture Content and Stability Studies:

The stability studies were performed in tri-laminated Aluminium pouches on one of the batches of example 6 and results are tabulated in Table 1 below:

TABLE 1
Results of Accelerated and room temperature studies in packed condition

| | | Accelerated | Accelerated | Accelerated | Room | Room |
|---|---|---|---|---|---|---|
| | | study | study | study | Temperature 1 | Temperature 2 |
| | | 1 M, | 3 M, | 6 M, | 6 M, | 6 M, |
| | | 40° C./ | 40° C./ | 40° C./ | 30° C./ | 25° C./ |
| Parameter | Initial | 75% RH | 75% RH | 75% RH | 75% RH | 60% RH |
| Description | white to off | white to off | white to off | white to off | white to off | white to off |
| | white colored | white colored | white colored | white colored | white colored | white colored |
| | powder | powder | powder | powder | powder | powder |
| Texture | Free Flowing | Free Flowing | Free Flowing | Free Flowing | Free Flowing | Free Flowing |
| | powder | powder | powder | powder | powder | powder |
| Visual | No lumps | No lumps | No lumps | No lumps | No lumps | No lumps |
| Observation | observed | observed | observed | observed | observed | observed |
| Odour | Characteristic | Characteristic | Characteristic | Characteristic | Characteristic | Characteristic |
| Loss on | 2.02% | 8.02% | 10.78% | 8.20% | 11.02% | 6.70% |
| Drying | | | | | | |
| Nitrite | 18750 | 18350 | Not Done | 17950 | 18250 | 18200 |
| content | | | | | | |

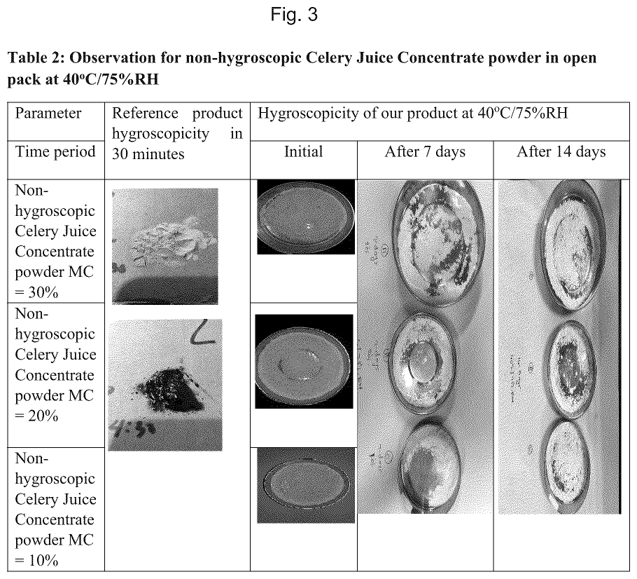

Based on the observations of stability as shown in Table 1 and FIG. 3 (Table 2), it was seen that the product does not absorb any moisture when exposed to higher inherent moisture level and % Relative humidity

Analysis of the physicochemical properties revealed that appearance, flow properties, hygroscopicity, moisture content and water dispersibility were not affected significantly during the accelerated stability studies.

Since a water insoluble inorganic salt is used as the carrier for the curing agent, the uniform dispersion in water can be filtered and the filtrate comprising the curing agent can be used directly as a curing agent by spraying the filtrate directly onto meat products or use of the filtrate directly in the food depending on the desired concentration.

Since the powder is non-hygroscopic, there is no risk of water absorption and subsequent discoloration and liquefaction of the product once a pack is opened. In addition, irrespective of the variations that occur during storage, transportation and use the product remains stable.

Analysis of the nitrite content in samples: AOAC Official Method 935.48, AOAC Official Method 993.03 are the methods that were used to analyse the nitrite content in the samples. Since the curing effect is mediated through the nitrite content in celery powder, we analysed the nitrite content in the concentrated liquid Celery Juice Concentrate which was the input material and the nitrite content in the finished product which is the stable non-hygroscopic powdered Celery Juice Concentrate and results obtained are given below:

TABLE 3
Nitrite content of Celery non-hygroscopic powder

| Non-hygroscopic celery | | Theoretically |
|---|---|---|
| extract granules/Powder | | expected |
| | Actual Results | Results |
| Batch No. | (Nitrite in ppm) | (Nitrite in ppm) |
| Example 1 | 23452 | 22500 |
| Example 2 | 27789 | 27000 |
| Example 3 | 15369 | 15000 |
| Example 4 | 22000 | 22058 |
| Example 5 | 18480 | 18500 |
| Example 6 | 18750 | 18000 |
| Example 7 | 16575 | 15000 |
| Example 8 | 24825 | 24800 |
| Example 9 | 24750 | 24500 |
| Example 10 | 15210 | 15600 |

Thus, it can be seen that the nitrite content is well protected and the actual results obtained are within ±10.0%, preferably within ±5.0%, of theoretical values while processing Celery extract liquid concentrate to powder and/or granular form.

1. A formulation comprising a powdered non-hygroscopic Celery juice concentrate, precipitated Silica and its salts or an inorganic water insoluble metal ion salt and a diluent.

2. A formulation of claim 1, wherein the nitrite content is between 12500 to 30000 ppm.

3. A formulation of claim 1 wherein the ratio of the precipitated Silica to the concentrated liquid extract varies from 1:1 to 1:20, more preferably from 1:0.5 to 1:10.

4. A formulation of claim 1 wherein the ratio of the silica salt/water insoluble inorganic metal ion salt to the concentrated liquid extract varies from 1:1 to 1:10.

5. A formulation of claim 1 wherein the ratio of the concentrated liquid extract to the silica salt/water insoluble inorganic metal ion salt varies from 0.1:1 to 1:1.

6. A formulation of claim 1 wherein the diluent is a hydrophobic ingredient or a polymerised cellulose derivative or a binder.

7. A formulation of claim 1 wherein the water insoluble inorganic silica-based salt is Calcium Silicate, Magnesium Silicate, Sodium Tri Silicate and the like.

8. A formulation of claim 1 wherein the water insoluble inorganic metal ion-based salt is Magnesium Citrate and the like.

9. A formulation of claim 1 wherein the diluent is a hydrophobic ingredient functioning as a lubricant in a concentration of 0.01 to 0.05% w/w, like silicon dioxide, magnesium stearate, talc and the like.

10. A formulation of claim 1 wherein the diluent is a polymerised cellulose derivative like starch/modified starch or cellulose derivatives or polymeric carbohydrates in a concentration from 2 to 15% w/w, preferably between 4 to 12% w/w.

11. A formulation of claim 1 wherein the diluent is a binder like cellulose or its derivatives like HPMC in a concentration from 0.5 to 15% w/w, more preferably from 1 to 10% w/w.

12. A formulation of claim 1 wherein the diluent is a hydrophobic oil ingredient like Sunflower oil or a Lecithin based oil in a concentration from 1.0 to 25% w/w, more preferably from 7 to 22% w/w.

13. A formulation of claim 1, having a bulk density of at least 0.25 g/cc.

14. A formulation of claim 1, having a particle size distribution of d 10, d50 and d90 and particle volume diameter.

15. A formulation of claim 1, wherein the composition when exposed to room temperature does not absorb moisture by more than 20% w/w and is easily reconstituted to form a dispersion.

16. A process for the manufacture of a granular powdered non-hygroscopic Celery Juice Concentrate from a concentrated liquid extract of Celery as a plant material, comprising the steps of:
a) Addition of the Celery juice concentrate in full or parts, in the form of a solution/suspension to precipitated Silica or its salts or to a water insoluble inorganic metal ion salt.
b) Granulation of the wet mass of step a) at a suitable speed and time to obtain granules.
c) Addition of a diluent followed by mixing and blending.
Optionally, heating the mass in the blender or using a suitable dryer at a temperature ranging from 15° C. to 150° C., to remove moisture and achieve a desired moisture content.
